(19) United States
(12) Patent Application Publication
(10) Pub. No.: US 2024/0315579 A1
(43) Pub. Date: Sep. 26, 2024

(54) COVERS FOR CUFF BLOOD PRESSURE MEASUREMENTS

(71) Applicants: King-Wah Walter Yeung, Cupertino, CA (US); Wei-Wei Vivian Yeung, Cupertino, CA (US)

(72) Inventors: King-Wah Walter Yeung, Cupertino, CA (US); Wei-Wei Vivian Yeung, Cupertino, CA (US)

(21) Appl. No.: 18/543,239

(22) Filed: Dec. 18, 2023

Related U.S. Application Data
(60) Provisional application No. 63/454,278, filed on Mar. 23, 2023.

(51) Int. Cl. A61B 5/021; A61B 90/00

(52) U.S. Cl. CPC A61B 5/02141; A61B 90/08; A61B 2560/04; A61B 2560/06

(57) ABSTRACT

An inaccurate blood pressure (BP) measurement can result if a person is not thermally comfortable during a cuff BP measurement. For example, an inaccurate BP measurement can result if a person feels cold when his or her upper extremity that wears the BP measurement cuff is exposed to a low ambient temperature during a BP measurement. Although a person can wear thermally comfortable clothing, the person's upper extremity must be exposed to the ambient temperature during a cuff BP measurement. A cover, such as a fabric blanket, thermo-regulated blanket, electric blanket, plastic tube, etc., for a BP measurement cuff and/or for a person's upper extremity that wears the BP measurement cuff can improve the person's thermal comfort and can stabilize cuff BP measurements. In addition, warm or cool conditioned air with an appropriate level of humidity can be introduced into the cover to make the person even more thermally comfortable.

### CROSS-REFERENCE TO RELATED APPLICATIONS

This application claims the benefit of provisional application Ser. No. 63/454,278, filed 2023 Mar. 23 by the present inventors.

### BACKGROUND

### Prior Art

One of the most important routinely performed measurements at clinics or homes is blood pressure (BP) measurement with a BP measurement cuff. The measurement uses an inflatable BP measurement cuff that is wrapped around an extremity of a person, usually an upper arm. The cuff blood pressure measurement is performed by inflating the blood pressure measurement cuff to a pressure above the systolic pressure, and then the cuff pressure is reduced gradually. A sensor measures the cuff pressure, including the beat-to-beat pressure changes in the artery under the cuff, and the data is used to compute the systolic pressure, mean arterial pressure, and diastolic pressure. This method of measuring BP is discussed in U.S. Pat. No. 4,349,034 (1982) to Ramsey, III and U.S. Pat. No. 4,638,810 (1987) to Ramsey, III et al.

However, the accuracy of cuff BP measurement has long been a concern. In January 2023, the American Medical Association summarized a consensus document published in the Journal of Hypertension. The summary reports that cuff BP measurements performed in a clinical setting often lead to measurement errors that lead to inappropriate BP management decisions in 20% to 45% of cases. For example, just a 5 mm Hg BP measurement error can lead to inappropriate hypertension classification in 84 million individuals worldwide. The summary identifies four major causes of inaccuracy in cuff BP measurement, as discussed below.

The first major cause of cuff BP measurement inaccuracy is related to patients' habits or behaviors. For example, inappropriate timing and amount of meal ingestion, caffeine or nicotine use can all cause BP measurement inaccuracy. The summary also points out that a patient with a full bladder can lead to an error in systolic BP measurement of up to 33 mm Hg, and the white-coat effect can have an error of up to 26 mm Hg.

The second major cause of cuff BP measurement inaccuracy is related to the BP measurement conditions. For example, if a patient's arm is lower than heart level, the BP measurement error could be 4 mm Hg to 23 mm Hg. In addition, BP measurement error results if the patient's legs are crossed at the knees, if the patent talks during BP measurement, or if the BP measurement cuff pressure is reduced too rapidly.

The third major cause of cuff BP measurement inaccuracy is related to the BP measurement equipment. BP measurement errors can occur if the BP measurement cuff is too small or too large, or an unvalidated automated BP measurement device is used.

The fourth major cause of cuff BP measurement inaccuracy is related to errors in administering the BP measurement by physicians or health professionals. Common errors in a clinical setting include following a two-minute casual procedure to take BP measurement instead of following a standard procedure of about eight minutes (including a five-minute rest period prior to BP measurement) and failure to take multiple BP measurements.

Many people have higher blood pressure in a clinical setting compared to the blood pressure measured at home. This is known as white-coat hypertension, white-coat syndrome, or white-coat effect. As discussed in paragraph above, the white-coat effect can cause a large BP measurement error of up to 26 mm Hg higher in a clinical setting, and the effect tends to be greater in older patients, in women, and in patients with high BP (i.e., hypertension). The white-coat effect is thought to be caused by the anxiety of being around doctors in white coats in a clinical setting. The anxiety triggers a reaction in the sympathetic nervous system, which elevates the BP.

Usually, physicians do not prescribe medication to treat high BP based on just one high BP measurement in their clinics. Instead, a physician takes multiple BP measurements, or the patient takes multiple BP measurements at home with an automated BP measurement device to confirm that he or she does have high BP.

Cuff BP measurements have been the standard in clinical practice for many years. More recently, aortic BP, which requires an invasive procedure to measure, has been found to be clinically more relevant as an indicator of the health condition of the cardiovascular system. U.S. Pat. No. 10,835,132 B2 (2020) to Qasem discloses a method to compute the aortic BP wave from the cuff BP wave by using a calibration. However, the usefulness of this calibration relies heavily on the cuff BP wave measurement. Therefore, reducing the cuff BP measurement error also leads to improving the accuracy of the computed aortic BP wave.

### SUMMARY

To obtain an accurate cuff BP measurement, we have experimentally discovered that it is extremely important for a person or patient to be thermally comfortable during a cuff BP measurement. For example, an inaccurate cuff BP measurement can result if a person feels cold when his or her upper extremity that wears the BP measurement cuff is exposed to a low ambient temperature during a cuff BP measurement, especially when the duration of the cuff BP measurement is extended to accommodate for multiple BP measurements, or when the person has been wearing a warm long-sleeve before his or her upper extremity must be exposed to the low ambient temperature for the cuff BP measurement. The embodiments of the present invention use a cover for a BP measurement cuff and/or a person's upper extremity that wears the BP measurement cuff to improve the person's thermal comfort during a cuff BP measurement. The cuff BP measurement is performed after the cover is placed substantially or completely around the BP measurement cuff and/or the person's upper extremity that wears the BP measurement cuff for a predetermined duration. The predetermined duration is determined based on the specific design of the cover to make the person thermally comfortable during a cuff BP measurement. For example, when the cover is a fabric blanket with a certain thermal insulation property, the predetermined duration can be empirically made to approximately coincide with the person's rest period (e.g., five minutes) prior to the cuff BP measurement.

In accordance with a first embodiment of a cover for cuff BP measurement, the cover comprises a flexible overlay that wraps substantially or completely around the BP measurement cuff and a person's upper extremity that wears the BP measurement cuff. The flexible overlay can be a fabric blanket, thermo-regulated blanket, electric blanket, piece of leather, piece of paper, etc.

In accordance with a second embodiment of a cover for cuff BP measurement, the cover comprises a sleeve that is created with a part of a flexible overlay to cover a person's forearm and hand, and the rest of the flexible overlay (an upper-arm flexible overlay) is used to cover the BP measurement cuff and the person's upper arm that wears the BP measurement cuff. The flexible overlay can be a fabric blanket, thermo-regulated blanket, electric blanket, piece of leather, piece of paper, etc.

In accordance with a third embodiment of a cover for cuff BP measurement, the cover comprises a tie and a sleeve that is created with a part of a flexible overlay to cover a person's forearm and hand, and the rest of the flexible overlay (an upper-arm flexible overlay) is used to wrap substantially or completely around the BP measurement cuff and the person's upper arm that wears the BP measurement cuff. The tie is attached to the upper-arm flexible overlay to facilitate pulling the upper-arm flexible overlay to wrap substantially or completely around the BP measurement cuff and the person's upper arm. The flexible overlay can be a fabric blanket, thermo-regulated blanket, electric blanket, piece of leather, piece of paper, etc. The tie can be made of fabric, paper, metal foil (such as thin copper foil), etc., so long as the tie is flexible enough to wrap substantially or completely around the flexible overlay.

In accordance with a fourth embodiment of a cover for cuff BP measurement, the cover comprises a sleeve that is created with a part of a flexible overlay to cover the BP measurement cuff and a person's upper arm that wears the BP measurement cuff, and the rest of the flexible overlay (a forearm flexible overlay) is used to cover the person's forearm and hand. The flexible overlay can be a fabric blanket, thermo-regulated blanket, electric blanket, piece of leather, piece of paper, etc.

In accordance with a fifth embodiment of a cover for cuff BP measurement, the cover comprises a sleeve that is made with a flexible overlay or a tube to cover a person's forearm and hand. If the sleeve is made with a flexible overlay, then the flexible overlay can be a fabric blanket, thermo-regulated blanket, electric blanket, piece of leather, piece of paper, etc. If the sleeve is made with a tube, then the tube can be made of a material, such as plastic, rubber, paper, metal, etc., to make the person's forearm and hand thermally comfortable during a cuff BP measurement. In addition, warm or cool air (with an appropriate level of humidity, if necessary) can be introduced into the sleeve to make a person more thermally comfortable during the cuff BP measurement, and a flexible overlay (an upper-arm flexible overlay) can be connected to the sleeve to facilitate the conditioned air to flow to a person's upper arm and the BP measurement cuff as well.

In accordance with a sixth embodiment of a cover for cuff BP measurement, the cover comprises a sleeve that is made with a flexible overlay or a tube to cover the BP measurement cuff and a person's upper arm that wears the BP measurement cuff. If the sleeve is made with a flexible overlay, the flexible overlay can be a fabric blanket, thermo-regulated blanket, electric blanket, piece of leather, piece of paper, etc., to make the person's upper arm thermally comfortable during a cuff BP measurement. If the sleeve is made with a tube, then the tube can be made of a material, such as plastic, rubber, paper, metal, etc., to make the person's upper arm thermally comfortable during a cuff BP measurement. In addition, warm or cool air (with an appropriate level of humidity, if necessary) can be introduced into the sleeve to make the person more thermally comfortable during the cuff BP measurement, and a flexible overlay (a forearm flexible overlay) can be connected to the sleeve to facilitate the conditioned air to flow to the person's forearm and hand as well.

In accordance with a seventh embodiment of a cover for cuff BP measurement, the cover comprises a thermally comfortable material that is placed substantially or completely on the exterior surface of a BP measurement cuff. The thermally comfortable material can be made of rubber or thermal insulation coating, made with a flexible overlay (such as a fabric blanket, thermo-regulated blanket, electric blanket, piece of leather, piece of paper, etc.), etc., so long as the thermally comfortable material can make a person's upper arm thermally comfortable during a cuff BP measurement.

In accordance with an eighth embodiment of a cover for cuff BP measurement, the cover comprises of a long flexible overlay that wraps substantially or completely around the BP measurement cuff and a person's upper extremity. The flexible overlay first wraps substantially or completely around a person's neck to provide support for further wrapping of the flexible overlay substantially or completely around the BP measurement cuff and the person's upper extremity that wears the BP measurement cuff. The flexible overlay can be a fabric blanket, thermo-regulated blanket, electric blanket, piece of leather, piece of paper, etc.

### DRAWINGS—FIGURES

FIG. 1 shows a flexible overlay that covers a part of a BP measurement cuff and a part of a person's upper extremity that wears the BP measurement cuff in accordance with the first embodiment.

FIG. 2 shows the flexible overlay in FIG. 1 when it is wrapped substantially or completely around the BP measurement cuff and the person's upper extremity that wears the BP measurement cuff in accordance with the first embodiment.

FIG. 3 shows a flexible overlay before a part of it is used to create a sleeve for covering a person's forearm and hand in accordance with the second embodiment.

FIG. 4 shows the flexible overlay in FIG. 3 after a part of it is used to create a sleeve for covering a person's forearm and hand in accordance with the second embodiment.

FIG. 5 shows the flexible overlay in FIG. 4 after a person's forearm and hand are inserted into the sleeve of the flexible overlay, with a part of a BP measurement cuff and a part of the person's upper arm that wears the BP measurement cuff covered by the rest of the flexible overlay (an upper-arm flexible overlay) in accordance with the second embodiment.

FIG. 6 shows the flexible overlay in FIG. 5 after the BP measurement cuff and the person's upper arm that wears the BP measurement cuff are also covered by wrapping the rest of the flexible overlay (the upper-arm flexible overlay) substantially or completely around them in accordance with the second embodiment.

FIG. 7 shows a flexible overlay with a tie attached before a part of the flexible overlay is used to create a sleeve for covering a person's forearm and hand in accordance with the third embodiment.

FIG. 8 shows the flexible overlay and the tie in FIG. 7 after a part of the flexible overlay is used to create a sleeve for covering a person's forearm and hand in accordance with the third embodiment.

FIG. 9 shows the flexible overlay in FIG. 8 after a person's forearm and hand are inserted into the sleeve of the flexible overlay, with a part of a BP measurement cuff and a part of the person's upper arm that wears the BP measurement cuff covered by the rest of the flexible overlay (an upper-arm flexible overlay) in accordance with the third embodiment.

FIG. 10 shows the flexible overlay in FIG. 9 after the BP measurement cuff and the person's upper arm that wears the BP measurement cuff are also covered by pulling the tie to wrap the rest of the flexible overlay (the upper-arm flexible overlay) substantially or completely around them in accordance with the third embodiment.

FIG. 11 shows a flexible overlay before a part of it is used to create a sleeve for covering a BP measurement cuff and a person's upper arm that wears the BP measurement cuff in accordance with the fourth embodiment.

FIG. 12 shows the flexible overlay in FIG. 11 after a part of it is used to create a sleeve for covering a BP measurement cuff and a person's upper arm that wears the BP measurement cuff in accordance with the fourth embodiment.

FIG. 13 shows the flexible overlay in FIG. 12 after the sleeve of the flexible overlay is worn on a person's upper arm in accordance with the fourth embodiment.

FIG. 14 shows the flexible overlay in FIG. 13 after the sleeve of the flexible overlay is rolled up to the top of the person's upper arm to accommodate for wearing a BP measurement cuff on the person's upper arm in accordance with the fourth embodiment.

FIG. 15 shows the flexible overlay in FIG. 14 after a BP measurement cuff is worn on the person's upper arm in accordance with the fourth embodiment.

FIG. 16 shows the flexible overlay in FIG. 15 after the sleeve of the flexible overlay is rolled down to cover the BP measurement cuff and the person's upper arm, with parts of the person's forearm and hand covered by the rest of the flexible overlay (a forearm flexible overlay), in accordance with the fourth embodiment.

FIG. 17 shows the flexible overlay in FIG. 16 after the person's forearm and hand are also covered by wrapping the rest of the flexible overlay (the forearm flexible overlay) substantially or completely around them in accordance with the fourth embodiment.

FIG. 18 shows a BP measurement cuff that is worn on a person's upper arm and a sleeve that covers the person's forearm and hand, in accordance with the fifth embodiment.

FIG. 19 shows a sleeve that covers a BP measurement cuff (only the cuff tubing is shown, because the cuff is covered) and a part of a person's upper arm that wears the BP measurement cuff in accordance with the sixth embodiment.

FIG. 20 shows a thermally comfortable material that is placed substantially or completely on the exterior surface of a BP measurement cuff (only the cuff tubing is shown, because the cuff is covered with the thermally comfortable material), which is worn on a person's upper arm, in accordance with the seventh embodiment.

FIG. 21 shows a long flexible overlay that wraps substantially or completely around a person's upper extremity and a BP measurement cuff (only the cuff tubing is shown, because the cuff is covered by the long flexible overlay) that is worn on the person's upper arm in accordance with the eighth embodiment.

### DETAILED DESCRIPTION

We believe that a relatively low ambient temperature, which is usually between 70° F. (21° C.) and 75° F. (24° C.) in a clinical setting, is one of the major causes of the white-coat effect. The average normal body temperature is much higher at 98.6° F. (37° C.), which varies from 97° F. (36.1° C.) to 99° F. (37.2° C.), depending on the person, age, activity, and time of the day. A patient could feel cold when he or she walks into a clinical setting from a relatively warm outdoor environment. In addition, during a cuff BP measurement, a patient could feel cold when his or her upper extremity is exposed directly (or through very thin clothing) to the relatively low ambient temperature of a clinical setting, or when a cold BP measurement cuff makes physical contact directly (or through very thin clothing) with the patient's upper arm. The patient's cold feeling could trigger complex local (e.g., local vasoconstriction) and systemic (e.g., autonomic) thermoregulation responses, which we believe contribute to the white-coat effect, especially when the measurement duration is extended to accommodate for multiple cuff BP measurements, or when a patient has been wearing a warm long-sleeve before his or her upper extremity must be exposed to a low ambient temperature for a cuff BP measurement.

It would be relatively easy to make a person's body thermally comfortable by wearing appropriate clothing, but the person's upper extremity must be exposed directly (or through very thin clothing) to the ambient temperature, and the person's upper extremity must make physical contact with a BP measurement cuff directly (or through very thin clothing) during a cuff BP measurement. The present invention uses a cover for a BP measurement cuff and/or for a person's upper extremity that wears the BP measurement cuff, to improve the person's thermal comfort during a cuff BP measurement. We have discovered that this cover stabilizes cuff BP measurements, likely because the cover reduces the magnitude and speed of the person's complex thermoregulation responses that impact a cuff BP measurement. The cover can be a flexible overlay (such as a fabric blanket, thermo-regulated blanket, electric blanket, piece of leather, piece of paper, etc.) or a tube that is made of a material, such as plastic, rubber, paper, metal, etc., that can make a person thermally comfortable during a cuff BP measurement. The cover can cover a person's entire upper extremity including the BP measurement cuff that is worn on it, only a part of a person's upper extremity (e.g., only the forearm), only a person's upper arm including the BP measurement cuff, only the BP measurement cuff, etc. In addition, a combination of the covers in two or more embodiments can be used together. The cover makes a person thermally comfortable during a cuff BP measurement when the ambient temperature is too low or too high in many situations, such as in a clinical setting, at home, outdoors, etc.

The cover can be made of many different materials in the embodiments of the present invention. When the cover is a flexible overlay, it can be a blanket, such as a fabric blanket, thermo-regulated blanket, electric blanket, etc. The fabric blanket can be made of any fabric, such as polyester, cotton, wool, etc., or a combination of different types of fabrics. The fabric can be of any thickness, and a single layer or multiple layers of fabric can be used to create the fabric blanket. In addition, fabrics have been made that are capable of storing and dissipating heat energy during phase change through a variety of techniques, such as micro encapsulation and coating, etc. Hence, a thermo-regulated blanket can be made of phase-change thermo-regulated fabrics to improve a person's thermal comfort during a cuff BP measurement. One manufacturer of phase-change thermo-regulated fabrics is Outlast Technologies GmbH in Germany. The flexible overlay can also be made of other materials, such as leather, paper, etc., so long as the flexible overlay can make a person thermally comfortable during a cuff BP measurement. When the cover comprises a sleeve, warm or cool air (with an appropriate level of humidity, if necessary) can be introduced into the sleeve (such as through a pipe) with an advanced thermal control (such as that which is used for air conditioning) to make a person thermally comfortable during a cuff BP measurement. Heating elements, such as heat wires, can also be incorporated in the sleeve to make a person thermally comfortable.

The cover can also be made with an electric blanket or an electric heating pad, which is a miniature electric blanket. A common electric blanket is made with heat wires that are coiled within a fabric blanket. For example, various miniature electric blankets and electric heating pads are manufactured by Comfheat in China, and they are available through distributors in the USA. Advanced carbon-fiber resistive heating blankets can be thermo-regulated more easily because of the positive temperature coefficient in resistivity of the more evenly distributed carbon fibers in the blanket. In addition, a novel blanket made with a rubber-like base polymer loaded with specially blended conductive particles also exhibits a positive temperature coefficient in resistivity. By regulating the applied voltage, this novel thermo-regulated blanket can be heated to a pre-defined temperature, depending on the product, power source, and system configuration. One manufacturer of this novel blanket is EXO2 in Georgia.

The following embodiments disclose using a cover for cuff BP measurement performed on the left upper arm of a person. However, using a cover for cuff BP measurement with the cuff worn on the left upper arm for the embodiments should not be construed as limiting the scope of the embodiments, but merely as providing illustrations of some of the presently preferred embodiments. For example, the cuff BP measurement can be performed with a BP measurement cuff worn on the right upper arm, or with the cuff worn on the left or right wrist, without departing from the spirit and the scope of the embodiments. Furthermore, the following description of the embodiments is not intended to limit the invention to these embodiments, but rather to enable any person skilled in the art to make and use the invention. For example, the following embodiments for cuff BP measurement performed on the left upper arm can be modified by one skilled in the art to embodiments for cuff BP measurement performed on the right upper arm by making left-to-right mirror images of the following embodiments.

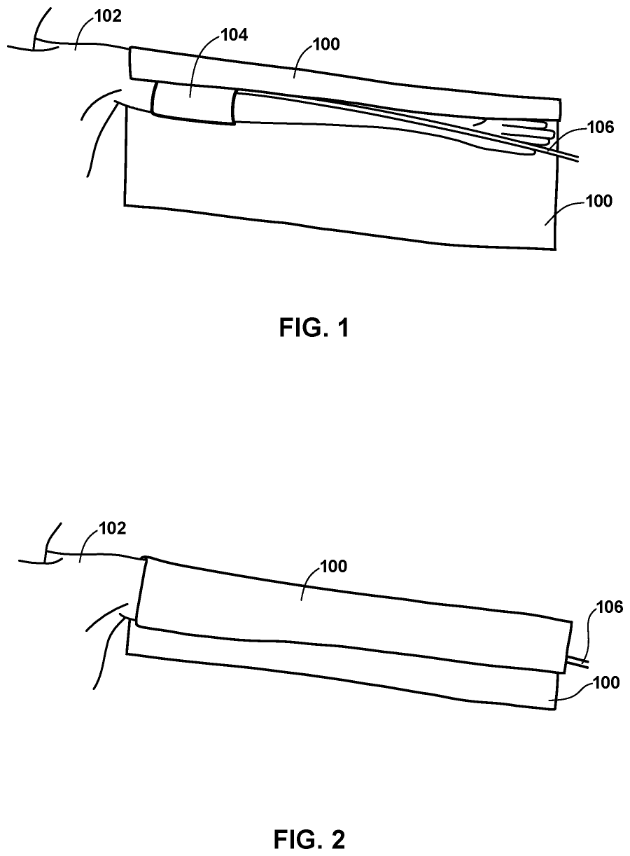

### FIGS. 1 and 2—First Embodiment

A first embodiment of a cover for cuff BP measurement is illustrated in FIGS. 1 and 2. In FIG. 1, the cover is flexible overlay 100, which covers a part of the upper extremity of person 102 and a part of BP measurement cuff 104. A part of flexible overlay 100 hangs down from the upper extremity of person 102, and cuff tubing 106 of BP measurement cuff 104 is supported by the hand of person 102. FIG. 2 shows that flexible overlay 100 wraps substantially or completely around the BP measurement cuff and the upper extremity of person 102 that wears the BP measurement cuff, and the excess part of flexible overlay 100 hangs down from the upper extremity of person 102. A section of cuff tubing 106 is also wrapped around by flexible overlay 100, so that cuff tubing 106 no longer needs the support of the hand. Although an excess part of flexible overlay 100 is shown to hang down from the upper extremity of person 102 in FIG. 2, a smaller flexible overlay might not have any excess part to hang down after the flexible overlay wraps substantially or completely around the upper extremity of person 102 and the BP measurement cuff.

Although FIG. 2 shows that cuff tubing 106 exits from one end of flexible overlay 100 near the hand of person 102, cuff tubing 106 can exit from flexible overlay 100 in different ways, especially if the flexible overlay is large enough to wrap substantially or completely around the upper extremity of person 102 and BP measurement cuff 104 in other ways. Flexible overlay 100 can be a fabric blanket that is made of a fabric, such as polyester, cotton, wool, etc., of any thickness, or a combination of different types of fabrics of any thickness, and a single or multiple layers of fabric can be used. If necessary, one or more fabric fasteners (e.g., safety pins, buttons, hook-and-loop fasteners such as Velcro®, etc.) can be used to secure flexible overlay 100 in place. Flexible overlay 100 can also be a thermo-regulated blanket, electric blanket, piece of leather, piece of paper, etc.

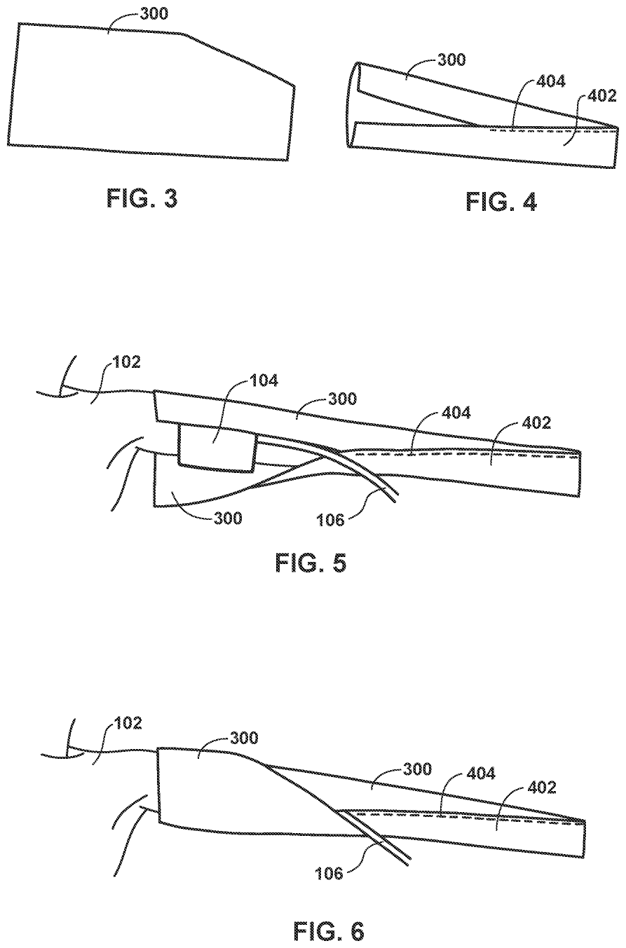

### FIGS. 3, 4, 5, and 6—Second Embodiment

A second embodiment of a cover for cuff BP measurement is illustrated in FIGS. 3, 4, 5 and 6. FIG. 3 shows a shape of flexible overlay 300 before a part of it is used to create a sleeve for covering a person's forearm and hand. FIG. 4 shows flexible overlay 300 after a part of it is used to create sleeve 402 for covering a person's forearm and hand, and sleeve 402 is created by sewing along stitching line 404, assuming that flexible overlay 300 is made of a material, such as fabric or leather, that can be sewn. Stitching line 404 can follow any stitching paths (e.g., a path that travels above and below a straight line) without following a straight line as shown in FIG. 4. Alternatively, a flexible overlay of another shape (such as a rectangular shape) that is not cut into the shape of flexible overlay 300 shown in FIG. 3 can also be sewn into a sleeve similarly. If there is any extra uncut part of the flexible overlay, it can be left inside or outside the sleeve, or this extra uncut part of the flexible overlay can be attached to the sleeve by sewing, adhesives, safety pins, etc.

Flexible overlay 300 can be a fabric blanket, thermo-regulated blanket, electric blanket, piece of leather, piece of paper, etc. Instead of sewing a part of flexible overlay 300 to create sleeve 402 (as shown in FIG. 4), one or more fabric fasteners (e.g., safety pins, buttons, hook-and-loop fasteners such as Velcro®, etc.), adhesives, staples, etc., can be used to replace the sewing. In addition, a glove or mitten can be attached to the end of sleeve 402 (by sewing, fabric fasteners, adhesives, etc.) to accommodate for the hand and fingers of a person, or the end of sleeve 402 can simply be closed (by sewing, fabric fasteners, adhesives, etc.) to cover the hand and fingers of person 102 completely. Alternatively, person 102 can wear a glove or mitten that is not attached to sleeve 402 during the cuff BP measurement.

FIG. 5 shows flexible overlay 300 in FIG. 4 after the forearm and hand of person 102 are inserted into sleeve 402 (with stitching line 404) of flexible overlay 300, with a part of the upper arm of person 102 and a part of BP measurement cuff 104 covered by the rest of flexible overlay 300 (an upper-arm flexible overlay). A part of the rest of flexible overlay 300 (the upper-arm flexible overlay) is free to hang down from the upper arm of person 102, opening up flexible overlay 300. FIG. 6 shows flexible overlay 300 (with stitching line 404) in FIG. 5 after the upper arm of person 102 and the BP measurement cuff are covered with the free-hanging part of the upper-arm flexible overlay by wrapping it substantially or completely around them. Flexible overlay 300 also covers a section of cuff tubing 106, and cuff tubing 106 exits from flexible overlay 300 through a gap formed by the wrapping. If necessary, one or more pieces of adhesive magnetic tape (not shown in FIGS. 3, 4, 5, and 6) can be attached to flexible overlay 300 to secure the wrapping. In FIGS. 5 and 6, sleeve 402 completely covers the forearm and hand of person 102, but sleeve 402 can cover only the forearm and a part of the hand, only the forearm, or only a part of the forearm of person 102, so long as person 102 feels thermally comfortable during the cuff BP measurement.

When compared with the first embodiment, the second embodiment provides a more stable cover for the upper extremity of person 102 and BP measurement cuff 104, and flexible overlay 300 can be smaller than flexible overlay 100. In addition, the second embodiment makes it easier for person 102 to wrap and stabilize flexible overlay 300 substantially or completely around the upper arm of person 102 and BP measurement cuff 104 with his or her other hand that is free.

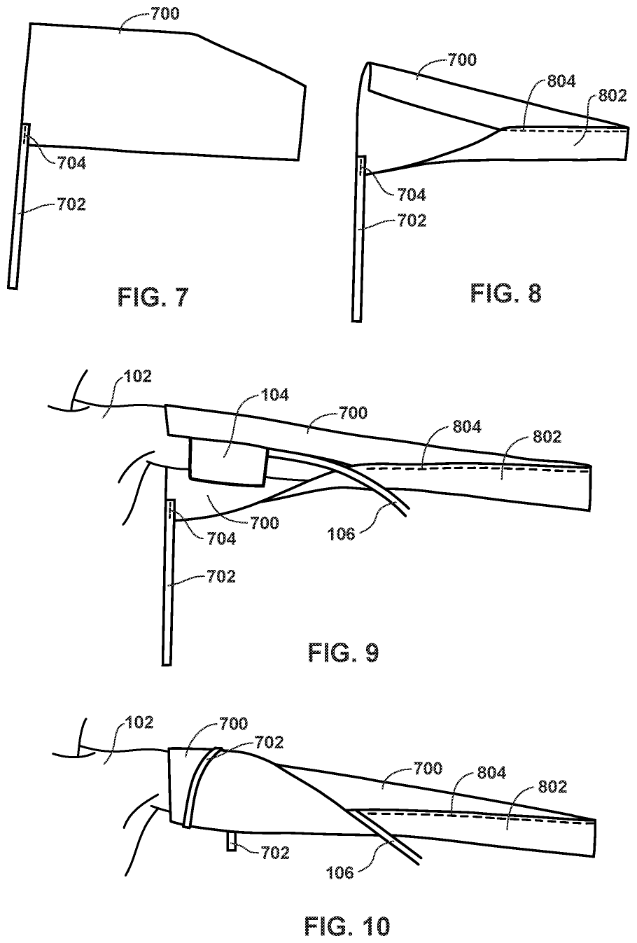

### FIGS. 7, 8, 9, and 10—Third Embodiment

A third embodiment of a cover for cuff BP measurement is illustrated in FIGS. 7, 8, 9 and 10. FIG. 7 shows a shape of flexible overlay 700 (with tie 702 attached) before a part of it is used to create a sleeve for covering a person's forearm and hand. FIG. 8 shows flexible overlay 700 (with tie 702 attached) after a part of it is used to create sleeve 802 for covering a person's forearm and hand, and sleeve 802 is created by sewing along stitching line 804, assuming that flexible overlay 700 is made of a material, such as fabric or leather, that can be sewn. Stitching line 804 can follow any stitching paths (e.g., a path that travels above and below a straight line) without following a straight line as shown in FIG. 8. Alternatively, a flexible overlay of another shape (such as a rectangular shape) that is not cut into the shape of flexible overlay 700 can also be sewn into a sleeve similarly. If there is any extra uncut part of the flexible overlay, it can be left inside or outside the sleeve, or this extra uncut part of the flexible overlay can be attached to the sleeve by sewing, adhesives, safety pins, etc.

FIGS. 7 and 8 also show that tie 702 is attached to flexible overlay 700 to facilitate pulling of flexible overlay 700 to wrap substantially or completely around a BP measurement cuff and a person's upper arm that wears the BP measurement cuff. Tie 702 can be made of fabric, leather, paper, metal foil (such as thin copper foil), etc., so long as tie 702 is flexible enough to wrap substantially or completely around flexible overlay 700. Tie 702 stabilizes flexible overlay 700 if tie 702 can wrap substantially or completely around flexible overlay 700 after flexible overlay 700 wraps substantially or completely around a BP measurement cuff and a person's upper arm that wears the BP measurement cuff. In FIGS. 7 and 8, tie 702 is attached to flexible overlay 700 by sewing along stitching line 704. Stitching line 704 can follow any stitching paths (e.g., a rectangular path or a path that travels above and below a straight line) without following a straight line shown in FIGS. 7 and 8.

In FIGS. 7 and 8, tie 702 is shown to be a band (or a strip) that is sewn onto flexible overlay 700. Tie 702 can also be in another form, such as in the form of a ribbon, a string, a wire, etc., so long as tie 702 can facilitate pulling flexible overlay 700 to wrap substantially or completely around a BP measurement cuff and a person's upper arm that wears the BP measurement cuff. Instead of sewing, tie 702 can be attached to flexible overlay 700 by adhesives, stapling, clips, etc. When tie 702 is a band, a strip, or a ribbon made of a fabric (such as cotton, satin, or polyester, etc.), information about the manufacturer, the cover, the logo, etc., can be printed on the fabric band, strip, or ribbon. In addition, one or more fabric fasteners (e.g., safety pins, buttons, hook-and-loop fasteners such as Velcro®, etc.), adhesives, etc., can be used to attach tie 702 to flexible overlay 700 permanently or temporarily. Alternatively, tie 702 and flexible overlay 700 can be cut out from a single continuous piece of flexible overlay without the need to attach tie 702 to flexible overlay 700.

Flexible overlay 700 can be a fabric blanket, thermo-regulated blanket, electric blanket, piece of leather, piece of paper, etc. Instead of sewing a part of flexible overlay 700 to create sleeve 802 (as shown in FIG. 8), one or more fabric fasteners (e.g., safety pins, buttons, hook-and-loop fasteners such as Velcro®, etc.), adhesives, staples, etc., can be used to replace the sewing. In addition, a glove or mitten can be attached to the end of sleeve 802 (by sewing, fabric fasteners, adhesives, etc.) to accommodate for the hand and fingers of a person, or the end of sleeve 802 can simply be closed (by sewing, fabric fasteners, adhesives, etc.) to cover the hand and fingers of a person completely. Alternatively, a person can wear a glove or mitten that is not attached to sleeve 802 during the cuff BP measurement.

FIG. 9 shows flexible overlay 700 in FIG. 8 after the forearm and hand of person 102 are inserted into sleeve 802 (with stitching line 804) of flexible overlay 700, with a part of the upper arm of person 102 and a part of BP measurement cuff 104 covered by the rest of flexible overlay 700 (an upper-arm flexible overlay). Tie 702 (with stitching line 704) and a part of the rest of flexible overlay 700 (the upper-arm flexible overlay) are free to hang down from the upper arm of person 102, opening up flexible overlay 700. FIG. 10 shows flexible overlay 700 (with sleeve 802 and stitching line 804) in FIG. 9 after the upper arm of person 102 and the BP measurement cuff are covered by using tie 702 to pull the free-hanging part of the upper-arm flexible overlay to wrap substantially or completely around them. Tie 702 also wraps substantially or completely around flexible overlay 700 to keep flexible overlay 700 stable at the upper arm of person 102 and the BP measurement cuff. Although tie 702 is shown in FIG. 10 to have its free end to hang down after wrapping substantially or completely around flexible overlay 700, a shorter tie 702 might not have a free end to hang down after wrapping substantially or completely around flexible overlay 700. Flexible overlay 700 also covers a section of cuff tubing 106, which exits flexible overlay 700 through a gap formed by the wrapping. In FIGS. 9 and 10, sleeve 802 completely covers the forearm and hand of person 102, but sleeve 802 can cover only the forearm and a part of the hand, only the forearm, or only a part of the forearm of person 102, so long as person 102 feels thermally comfortable during the cuff BP measurement.

When compared with the second embodiment, the third embodiment provides an even more stable cover for the upper extremity of person 102 and BP measurement cuff 104, because tie 702 can be used to wrap substantially or completely around the part of flexible overlay 700 that covers the upper arm of person 102 and BP measurement cuff 104. The addition of tie 702 facilitates pulling of the upper-arm flexible overlay of flexible overlay 700 to wrap substantially or completely around the upper arm of person 102 and BP measurement cuff 104 by person 102 with his or her other hand that is free, or by someone who is assisting person 102 in the cuff BP measurement.

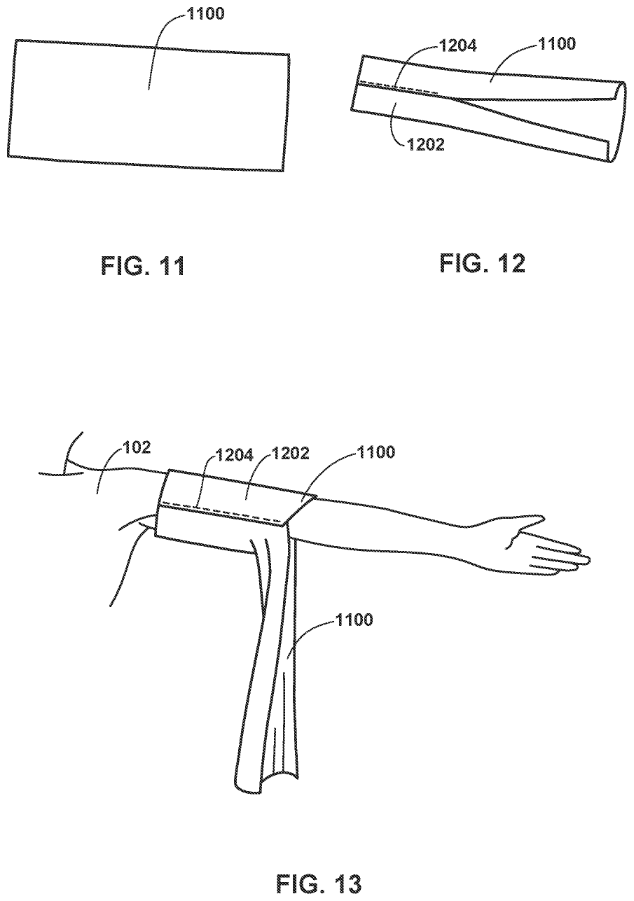

### FIGS. 11, 12, 13, 14, 15, 16, and 17—Fourth Embodiment

A fourth embodiment of a cover for cuff BP measurement is illustrated in FIGS. 11, 12, 13, 14, 15, 16, and 17. FIG. 11 shows a rectangular shape of flexible overlay 1100 before a part of it is used to create a sleeve for covering a BP measurement cuff and a person's upper arm that wears the BP measurement cuff. FIG. 12 shows flexible overlay 1100 in FIG. 11 after a part of it is used to create sleeve 1202 for covering a BP measurement cuff and a person's upper arm that wears the BP measurement cuff, and sleeve 1202 is created by sewing along stitching line 1204, assuming that flexible overlay 1100 is made of a material, such as fabric or leather, that can be sewn. FIG. 13 shows flexible overlay 1100 in FIG. 12 after sleeve 1202 (with stitching line 1204) of flexible overlay 1100 is worn on the upper arm of person 102, with the rest of flexible overlay 1100 (a forearm flexible overlay) hanging down from sleeve 1202.

Flexible overlay 1100 can be a fabric blanket, thermo-regulated blanket, electric blanket, piece of leather, piece of paper, etc. Stitching line 1204 can follow any stitching path (e.g., a path that travels above and below a straight line) without following a straight line as shown in FIGS. 12 and 13. Instead of sewing flexible overlay 1100 into sleeve 1202 (as shown in FIG. 12), one or more fabric fasteners (e.g., safety pins, buttons, hook-and-loop fasteners such as Velcro®, etc.), adhesives, staples, etc., can be used to replace the sewing.

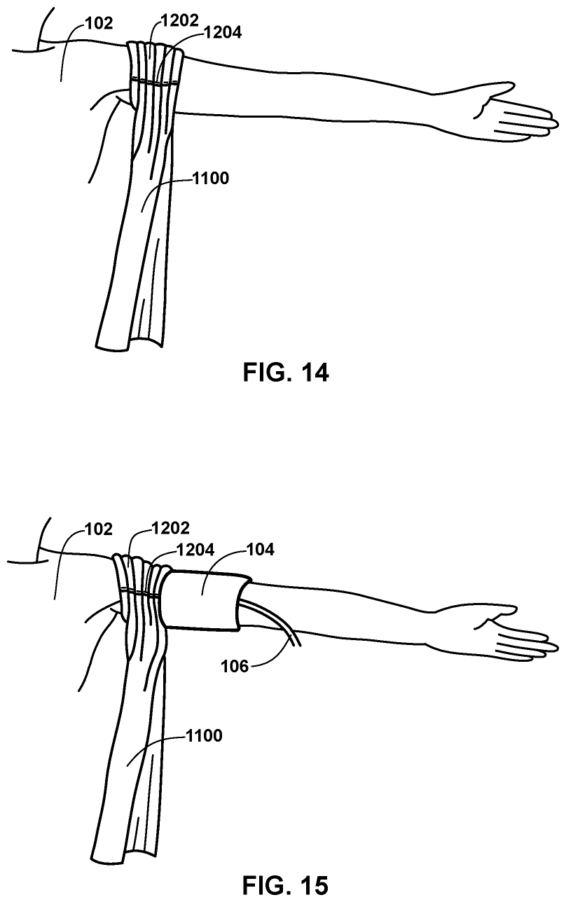

FIG. 14 shows sleeve 1202 (with stitching line 1204) of flexible overlay 1100 in FIG. 13 after sleeve 1202 is rolled up to the top of the upper arm of person 102 to accommodate for wearing a BP measurement cuff on the upper arm of person 102. FIG. 15 shows sleeve 1202 (with stitching line 1204) of flexible overlay 1100 in FIG. 14 after BP measurement cuff 104 (with cuff tubing 106) is worn on the upper arm of person 102. BP measurement cuff 104 is shown to squeeze sleeve 1202 a little when it is worn on the upper arm of person 102, although BP measurement cuff 104 might not need to squeeze sleeve 1202 if the cuff size is smaller or if a part of BP measurement cuff 104 slips underneath sleeve 1202.

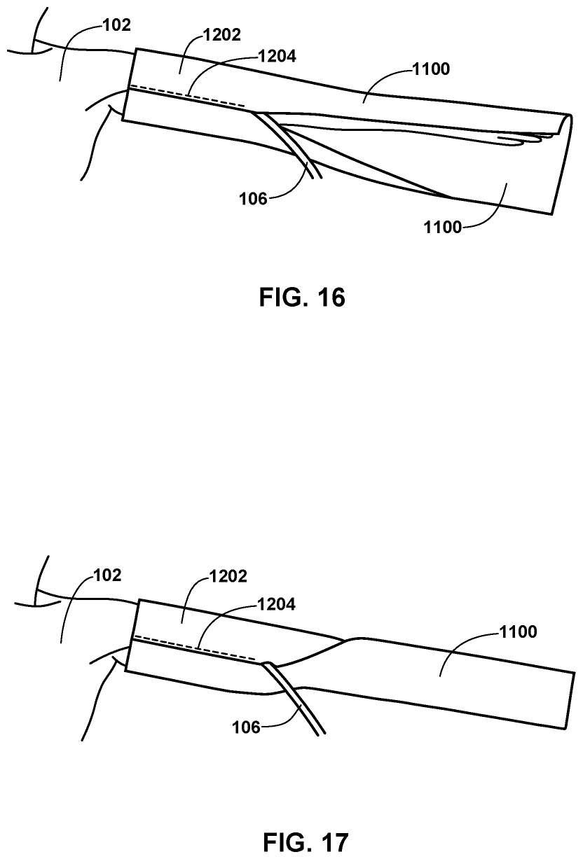

FIG. 16 shows flexible overlay 1100 in FIG. 15 after sleeve 1202 (with stitching line 1204) is rolled down to cover the upper arm of person 102 and the BP measurement cuff, so that only cuff tubing 106 is shown, and parts of the forearm and hand of person 102 are also covered by the rest of flexible overlay 1100 (the forearm flexible overlay). FIG. 17 shows flexible overlay 1100 (with sleeve 1202 and stitching line 1204) in FIG. 16 after the forearm and hand of person 102 are covered by wrapping the rest of flexible overlay 1100 (the forearm flexible overlay) substantially or completely around them. Cuff tubing 106 exits through a gap formed by the wrapping.

The fourth embodiment is simple to create, because only stitching along a single stitching line 1204 on a rectangular flexible overlay 1100 is needed. Instead of being rectangular, flexible overlay 1100 can also be of other shapes. The fourth embodiment facilitates person 102 to cover BP measurement cuff 104 and the upper extremity of person 102 that wears BP measurement cuff 104 with flexible overlay 1100 by using his or her other hand that is free, or by someone who is assisting the person in performing the cuff BP measurement. However, when compared to the second and the third embodiments, the fourth embodiment requires person 102 to meticulously remember to wear sleeve 1202 on his or her upper arm before wearing BP measurement cuff 104 on the upper arm, and it might be somewhat cumbersome to wear BP measurement cuff 104 after sleeve 1202 is already being worn on the upper arm, because sleeve 1202 tends to slip downwards to interfere with the wearing of BP measurement cuff 104.

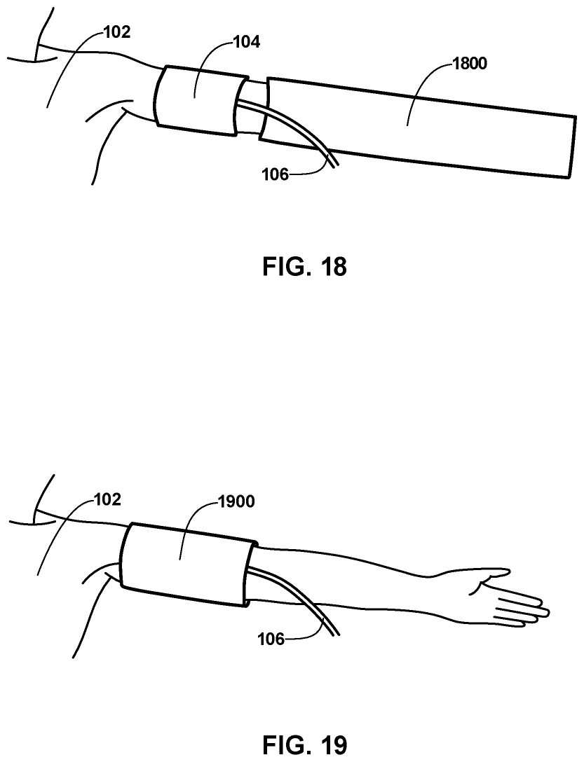

### FIG. 18—Fifth Embodiment

A fifth embodiment of a cover for cuff BP measurement is illustrated in FIG. 18. FIG. 18 shows that sleeve 1800 covers the forearm and hand of person 102, and BP measurement cuff 104 (with cuff tubing 106) is worn on the upper arm of person 102. Sleeve 1800 can be made with a flexible overlay such as a blanket, a tube, etc., so long as sleeve 1800 can make a person's forearm and hand thermally comfortable during a cuff BP measurement. If sleeve 1800 is a flexible overlay, it can be a fabric blanket, thermo-regulated blanket, electric blanket, piece of leather, piece of paper, etc. If the sleeve 1800 is a tube, it can be made of plastic, rubber, paper, metal, etc. Heating elements, such as heat wires, can also be incorporated in sleeve 1800 to make a person thermally comfortable.

Although sleeve 1800 does not cover the upper arm of person 102 and BP measurement cuff 104 to make the upper arm of person 102 thermally comfortable, sleeve 1800 is easily worn by person 102. Sleeve 1800 can incorporate an advanced thermal control, such as that which is used for air conditioning, to make the forearm and hand of person 102 even more thermally comfortable during the cuff BP measurement. In such a case, warm or cool air (with an appropriate level of humidity, if necessary) can be introduced to sleeve 1800 (e.g., through a pipe), and the conditioned air can flow from sleeve 1800 to the upper arm of person 102 and BP measurement cuff 104 to make the upper arm of person 102 more thermally comfortable as well. Sleeve 1800 can also be extended to cover the upper arm of person 102 and BP measurement cuff 104, with an appropriate opening (such as a slot) for cuff tubing 106 to exit sleeve 1800, or a separate cover can be used for the upper arm of person 102 and BP measurement cuff 104. Alternatively, a flexible overlay (an upper-arm flexible overlay) can be connected to sleeve 1800 to cover the upper arm of person 102 and BP measurement cuff 104. If conditioned air is used, this flexible overlay can also guide the conditioned air from sleeve 1800 to the upper arm of person 102 and BP measurement cuff 104 to make the upper arm of person 102 thermally comfortable.

Instead of leaving the end of sleeve 1800 near the hand of person 102 open, a glove or mitten can be attached to the end of sleeve 1800 (by using sewing, fabric fasteners, adhesives, etc.) to accommodate for the hand and fingers of person 102, or the end of sleeve 1800 can simply be closed (by sewing, fabric fasteners, adhesives, etc.) to cover the hand and fingers of person 102 completely. Alternatively, person 102 can wear a glove or mitten that is not attached to sleeve 1800 during the cuff BP measurement. In FIG. 18, sleeve 1800 completely covers the forearm and hand of person 102, but sleeve 1800 can cover only the forearm and a part of the hand, only the forearm, or only a part of the forearm of person 102, so long as person 102 feels thermally comfortable during the cuff BP measurement.

### FIG. 19—Sixth Embodiment

A sixth embodiment of a cover for cuff BP measurement is illustrated in FIG. 19. FIG. 19 shows that sleeve 1900 covers the upper arm of person 102 and the BP measurement cuff (the cuff is completely covered by sleeve 1900, so that only cuff tubing 106 is shown). Sleeve 1900 can be made with a flexible overlay, a tube, etc., so long as sleeve 1900 can make a person's upper arm thermally comfortable during a cuff BP measurement.

If sleeve 1900 is made with a flexible overlay, the flexible overlay can be a fabric blanket, thermo-regulated blanket, electric blanket, piece of leather, piece of paper, etc., and buttons, hook-and-loop fasteners such as Velcro®, etc., can be used to form sleeve 1900 after the flexible overlay substantially or completely covers the upper arm of person 102 and the BP measurement cuff. Alternatively, sleeve 1900 can be created with the same technique as that for sleeve 1202 (as shown in FIG. 12) in the fourth embodiment (but without the rest of flexible overlay 1100 for covering the forearm and hand), and sleeve 1900 can be worn to cover the upper arm of person 102 and the BP measurement cuff with the steps illustrated for the fourth embodiment (as shown in FIGS. 13, 14, 15, and 16). If sleeve 1900 is a tube, it can be made of a material, such as plastic, rubber, paper, metal, etc., that can make the upper arm of person 102 thermally comfortable, and heating elements (such as heat wires) can also be incorporated in sleeve 1900. An appropriate cutout (such as a slot) on sleeve 1900 can facilitate cuff tubing 106 exiting sleeve 1900 when sleeve 1900 slides up from the forearm to cover the upper arm of person 102 and the BP measurement cuff. Alternatively, an elastic tube (such as a rubber tube) can be cut, so that it can be opened up to facilitate the upper arm of person 102 and the BP measurement cuff to enter the inside of the tube, and then the elastic tube can return to its original shape (i.e., the shape of a tube) of sleeve 1900 afterward.

Although sleeve 1900 does not cover the forearm and hand of person 102 to make the forearm and hand of person 102 thermally comfortable, sleeve 1900 is relatively easily worn by person 102. Sleeve 1900 can incorporate an advanced thermal control, such as that which is used for air conditioning, to make the upper arm of person 102 more thermally comfortable during the cuff BP measurement. In such a case, warm or cool air (with an appropriate level of humidity, if necessary) can be introduced into sleeve 1900 (such as through a pipe), and the conditioned air can flow from sleeve 1900 to make the forearm and hand of person 102 thermally comfortable as well. In addition, a flexible overlay (a forearm flexible overlay) can be connected to sleeve 1900 to cover the forearm and hand of person 102. If conditioned air is used, this flexible overlay can also guide the conditioned air from sleeve 1900 to the forearm and hand of person 102 to make the forearm and hand of person 102 thermally comfortable.

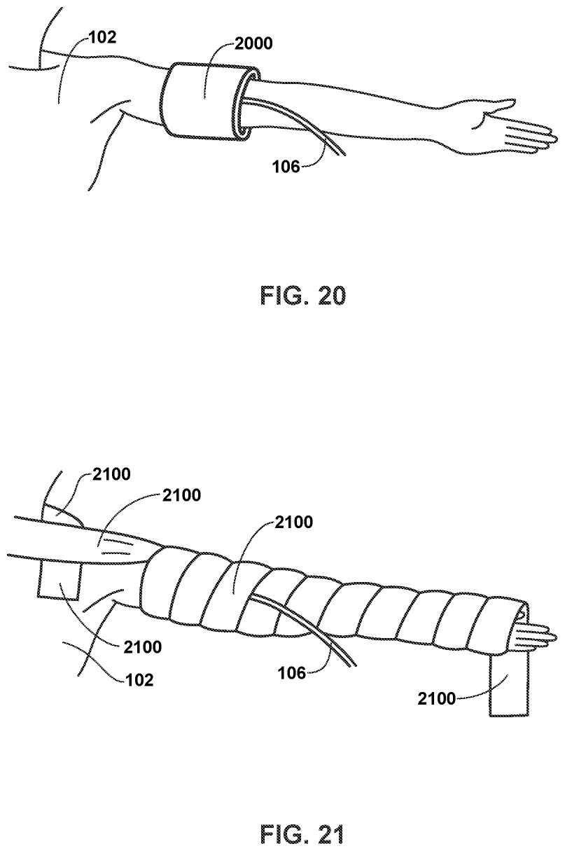

### FIG. 20—Seventh Embodiment

A seventh embodiment of a cover for cuff BP measurement is illustrated in FIG. 20. FIG. 20 shows that thermally comfortable material 2000 not only covers the BP measurement cuff (and the part of the upper arm of person 102 underneath the BP measurement cuff), but also extends out to cover an additional part of the upper arm of person 102, so that only cuff tubing 106 is shown. Thermally comfortable material 2000 can be placed substantially or completely on the exterior surface of a BP measurement cuff to cover a part of the upper arm that is underneath the BP measurement cuff, or can be extended out from the BP measurement cuff to cover an additional part of the upper arm of person 102 as well (as shown in FIG. 20). The purpose of thermally comfortable material 2000 is to make the upper arm of person 102 thermally comfortable, and a sleeve that is similar to sleeve 1800 (as shown in FIG. 18) in the fifth embodiment can be added to make the forearm and hand of person 102 thermally comfortable as well.

Thermally comfortable material 2000 can be made with a flexible overlay, such as a fabric blanket, thermo-regulated blanket, electric blanket, piece of leather, piece of paper, etc., so long as thermally comfortable material 2000 can make the upper arm of person 102 thermally comfortable during a cuff BP measurement. Thermally comfortable material 2000 can also be made of a material, such as rubber, thermal insulation coating, etc., to provide thermal insulation for the upper arm of person 102 and the BP measurement cuff from the ambient temperature, which might be too cold or too hot for person 102.

Thermally comfortable material 2000 can be placed substantially or completely on the exterior surface of a BP measurement cuff during the manufacturing process of the BP measurement cuff by attaching with coating, sewing, adhesives, or hook-and-loop fasteners such as Velcro®, etc. Alternatively, the user can place thermally comfortable material 2000 substantially or completely on the exterior surface of a BP measurement cuff by attaching with sewing, adhesives, safety pins, or hook-and-loop fasteners such as Velcro®, etc. Furthermore, thermally comfortable material 2000 can be placed substantially or completely on the exterior surface of a BP measurement cuff without any attachment. For example, thermally comfortable material 2000 can be a flexible overlay such as a fabric blanket that can be placed substantially or completely on the exterior surface of a BP measurement cuff of a public blood-pressure kiosk machine in pharmacies without any attachment.

### FIG. 21—Eighth Embodiment

An eighth embodiment of a cover for cuff BP measurement is illustrated in FIG. 21. FIG. 21 shows that long flexible overlay 2100 wraps around the BP measurement cuff (which is worn on the upper arm) and most of the upper extremity of person 102, so that only cuff tubing 106 is shown. Although flexible overlay 2100 is shown to first wrap substantially or completely around the neck of person 102 to provide support to flexible overlay 2100 for further wrapping flexible overlay 2100 substantially or completely around the upper extremity of person 102 and the BP measurement cuff, flexible overlay 2100 does not have to wrap substantially or completely around the neck of person 102, especially if someone can help person 102 wrap flexible overlay 2100 substantially or completely around his or her upper extremity and the BP measurement cuff. Alternatively, one end of a long flexible overlay can form a loop by sewing, safety pins, button, hoop-and-loop fasteners such as Velcro®, etc., and the loop can be worn around the neck of person 102 to provide support before the rest of the long flexible overlay 2100 wraps substantially or completely around the BP measurement cuff and the patient's upper extremity that wears the BP measurement cuff. Flexible overlay 2100 can be a fabric blanket, thermo-regulated blanket, electric blanket, piece of leather, piece of paper, etc.

In FIG. 21, although flexible overlay 2100 wraps around the BP measurement cuff and the upper extremity (except for the fingers, a part of the hand, and a part of the upper arm that is adjacent to the shoulder) of person 102, flexible overlay 2100 can wrap around the entire upper extremity (including the fingers, the hand, and the entire upper arm) of person 102 or only a small part of the upper extremity (e.g., only the forearm) of person 102. In addition, flexible overlay 2100 can wrap only around the BP measurement cuff, only around the BP measurement cuff and a part of the upper arm of person 102 that is adjacent to it, or only around a part of the upper extremity of person 102 that is not covered by the BP measurement cuff. If flexible overlay 2100 is used to wrap substantially or completely around the forearm and hand of person 102, the hand of person 102 can hold one end of flexible overlay 2100 to facilitate the wrapping of flexible overlay 2100 up from the hand of person 102 to substantially or completely cover the forearm and hand of person 102, or even further up to substantially or completely cover the upper arm of person 102 and the BP measurement cuff as well. The purpose of wrapping flexible overlay 2100 to cover the upper extremity of person 102 and the BP measurement cuff is to make person 102 thermally comfortable during a cuff BP measurement.

Although the description above contains many specificities, these should not be construed as limiting the scope of the embodiments, but as merely providing illustrations of some of the embodiments. For example, the above-described embodiments can be modified by one skilled in the art, such as by combining various described features or making left-to-right mirror images of the embodiments, without departing from the spirit and the scope of the embodiments. Thus the scope of the embodiments should be determined by the appended claims and their legal equivalents, rather than by the examples given.

We claim:

1. A method of improving a person's thermal comfort during a cuff blood pressure measurement using a blood pressure measurement cuff, comprising:
(a) providing a cover to be used for said cuff blood pressure measurement; and
(b) performing said cuff blood pressure measurement after said cover is placed substantially or completely around at least one of said blood pressure measurement cuff or said person's upper extremity that wears said blood pressure measurement cuff.

2. The method according to claim 1 wherein said cover is a flexible overlay.

3. The method according to claim 2 wherein the step of performing said cuff blood pressure measurement after said flexible overlay is placed substantially or completely around at least one of said blood pressure measurement cuff or said person's upper extremity that wears said blood pressure measurement cuff comprises performing said cuff blood pressure measurement after said flexible overlay is placed substantially or completely around said blood pressure measurement cuff and the upper arm of said person's upper extremity that wears said blood pressure measurement cuff.

4. The method according to claim 2 wherein the step of performing said cuff blood pressure measurement after said flexible overlay is placed substantially or completely around at least one of said blood pressure measurement cuff or said person's upper extremity that wears said blood pressure measurement cuff comprises performing said cuff blood pressure measurement after said flexible overlay is placed substantially or completely around the forearm and hand of said person's upper extremity that wears said blood pressure measurement cuff.

5. The method according to claim 2 wherein the step of performing said cuff blood pressure measurement after said flexible overlay is placed substantially or completely around at least one of said blood pressure measurement cuff or said person's upper extremity that wears said blood pressure measurement cuff comprises performing said cuff blood pressure measurement after said flexible overlay is placed substantially or completely around said blood pressure measurement cuff and said person's upper extremity that wears said blood pressure measurement cuff.

6. The method according to claim 2 wherein said flexible overlay is a blanket.

7. The method according to claim 1 wherein said cover is a thermally comfortable material that is placed substantially or completely on the exterior surface of said blood pressure measurement cuff, and the step of performing said cuff blood pressure measurement after said cover is placed substantially or completely around at least one of said blood pressure measurement cuff or said person's upper extremity that wears said blood pressure measurement cuff comprises performing said cuff blood pressure measurement after said thermally comfortable material on the exterior surface of said blood pressure measurement cuff is placed substantially or completely around the upper arm of said person's upper extremity that wears said blood pressure measurement cuff.

8. The method according to claim 1 wherein said cover is a sleeve.

9. The method according to claim 8 wherein the step of performing said cuff blood pressure measurement after said sleeve is placed substantially or completely around at least one of said blood pressure measurement cuff or said person's upper extremity that wears said blood pressure measurement cuff comprises placing said sleeve substantially or completely around the forearm and hand of said person's upper extremity that wears said blood pressure measurement cuff.

10. The method according to claim 8 further comprises introducing warm or cool air into said sleeve during said cuff blood pressure measurement.

11. A cover for improving a person's thermal comfort during a cuff blood pressure measurement using a blood pressure measurement cuff, comprising:
(a) a sleeve adapted to substantially or completely cover the forearm of said patient's upper extremity that wears said blood pressure measurement cuff; and
(b) an upper-arm flexible overlay connected to said sleeve and adapted to substantially or completely cover said blood pressure measurement cuff and said person's upper arm that wears said blood pressure measurement cuff.

12. The cover according to claim 11 wherein said sleeve is created with a part of a flexible overlay, and said upper-arm flexible overlay is the rest of said flexible overlay.

13. The cover according to claim 12 further comprises a tie attached to the rest of said flexible overlay and adapted to pull the rest of said cover flexible overlay to wrap substantially or completely around said blood pressure measurement cuff and said person's upper arm that wears said blood pressure measurement cuff.

14. The cover according to claim 12 wherein said flexible overlay is a fabric blanket.

15. The cover according to claim 14 further comprises a tie attached to the rest of said fabric blanket and adapted to pull the rest of said fabric blanket to wrap substantially or completely around said blood pressure measurement cuff and said person's upper arm that wears said blood pressure measurement cuff.

16. The cover according to claim 12 wherein said flexible overlay is an electric blanket.

17. The cover according to claim 12 wherein said flexible overlay is a thermo-regulated blanket.

18. A cover for improving a person's thermal comfort during a cuff blood pressure measurement using a blood pressure measurement cuff, comprising:
(a) a sleeve adapted to substantially or completely cover said blood pressure measurement cuff and said person's upper arm that wears said blood pressure measurement cuff; and
(b) a forearm flexible overlay connected to said sleeve and adapted to substantially or completely cover the forearm of said patient's upper extremity that wears said blood pressure measurement cuff.

19. The cover according to claim 18 wherein said sleeve is created with a part of a flexible overlay, and said forearm flexible overlay is the rest of said flexible overlay.

20. The cover according to claim 19 wherein said flexible overlay is a fabric blanket.
